(19) United States
(12) Patent Application Publication
(10) Pub. No.: US 2024/0200090 A1
(43) Pub. Date: Jun. 20, 2024

(54) Application of Phosphorus Starvation Response Factor PHR2 in Plant and Arbuscular Mycorrhizal Symbiosis and Improving Phosphorus Nutrition

(71) Applicant: CAS Center for Excellence in Molecular Plant Sciences, Shanghai (CN)

(72) Inventors: Ertao Wang, Shanghai (CN); Jincai Shi, Shanghai (CN); Boyu Zhao, Shanghai (CN); Xiaowei Zhang, Shanghai (CN); Huiling Dai, Shanghai (CN)

(21) Appl. No.: 18/288,069

(22) PCT Filed: Mar. 14, 2022
(86) PCT No.: PCT/CN2022/080694

§ 371 Date: Oct. 24, 2023

(30) Foreign Application Priority Data
Apr. 29, 2021 (CN) 202110477038.8

(51) Int. Cl. C12N 15/82; A01H 6/46; C12N 9/22; C12Q 1/6895; C12Q 1/6897

(52) U.S. Cl. CPC C12N 15/8261; A01H 6/46; C12N 9/22; C12N 15/8205; C12Q 1/6895; C12Q 1/6897; C12N 2310/20; C12Q 2600/13; C12Q 2600/158

(57) ABSTRACT

Provided is a method for regulating the symbiosis of gramineous plants and arbuscular mycorrhizal fungi or regulating the inhibitory effect of high phosphorus on mycorrhizal symbiosis, the method comprising: adjusting the expression or activity of the phosphorus starvation response factor PHR2 in gramineous plants. Also provided is the use of PHR2 or an encoding gene thereof in regulating the symbiosis of gramineous plants and arbuscular mycorrhizal fungi, regulating the inhibitory effect of high phosphorus on mycorrhizal symbiosis, and as a molecular marker for the identification of the symbiosis of gramineous plants and arbuscular mycorrhizal fungi. Also provided is a method for using PHR2 to screen for substances that regulate the symbiosis of gramineous plants and arbuscular mycorrhizal fungi or regulate the inhibitory effect of high phosphorus on mycorrhizal symbiosis. Also provided is a method for using PHR2 to identify mycorrhizal-associated genes regulated by PHR2 in mycorrhizal symbiosis.

### RELATED APPLICATIONS

This application is a National Stage Application under 35 U.S.C. 371 of co-pending PCT application PCT/CN2022/080694 designating the United States and filed Mar. 14, 2022; which claims the benefit of CN application number 202110477038.8 and filed Apr. 29, 2021, each of which are hereby incorporated by reference in their entireties.

### SEQUENCE LISTING

The instant application contains a Sequence Listing which has been submitted electronically in ANSI format and is hereby incorporated by reference in its entirety. Said ANSI copy, created on Oct. 24, 2023, is named “008703.00040_ST25” and is 6.01 KB in size.

### TECHNICAL FIELD

The disclosure belongs to the technical field of biotechnology and botany. More specifically, the disclosure relates to the application of phosphate starvation response factor PHR2 in plant and arbuscular mycorrhizal symbiosis, and the application in improving phosphorus nutrition.

### TECHNICAL BACKGROUND

Phosphorus is one of the three essential nutrients for plant growth and development. It is an important component of organisms in plants and participates in numerous physiological and biochemical processes of plants in various ways, including photosynthesis, respiration, biosynthesis, membrane structure, signal transduction and so on. It plays a vital role in life cycle of plants. Plants mainly acquire inorganic phosphorus in the form of phosphate (PO₄³⁻, HPO₄⁻ and H₂PO₄⁻). However, most of the phosphorus in soil exists as insoluble salts with organic phosphorus and positive ions such as Fe and Al, this reduces the solubility and mobility of phosphorus and limits the effective utilization of soil phosphorus by plants. Therefore, phosphorus becomes a major factor in limiting plant growth. In agricultural production, in order to ensure the yield of crops, a large amount of phosphate-containing fertilizers will be applied. However, most of the applied phosphate fertilizers are fixed in the soil and become organic phosphorus that cannot be used by plants and only about 30% are directly acquired and utilized by plants. A large amount of organic phosphorus in the soil also causes environmental problems such as water eutrophication and harmful algal bloom.

After a long-term evolution, most plants obtain phosphate from soil mainly through two ways: one is to directly acquire from soil through phosphate transporters, and the other is to establish a symbiotic relationship with mycorrhizal fungi, using mycorrhizal fungi to acquire phosphate. Mycorrhizal symbiosis is a symbiotic relationship between mycorrhizal fungi and most land plants, including important crops. Mycorrhizal symbiosis is also the oldest and most common symbiotic relationship in nature. Both plants and mycorrhizal fungi benefit from the symbiotic relationship: mycorrhizal fungi increase host plant availability of mineral nutrients, water uptake, disease resistance, and stress resistance; host plants provide mycorrhizal fungi with a carbon source for growth and reproduction.

In recent years, important progress has been made in the molecular mechanism of mycorrhizal symbiosis, but how plants balance their own phosphate uptake and the two ways of acquiring phosphate in arbuscular mycorrhizal fungi symbiosis is still unknown.

Therefore, it is necessary in this field to research in depth on the regulation of phosphate acquisition in arbuscular mycorrhizal symbiosis, so as to clarify the pathway of phosphate acquisition of plants in symbiosis with arbuscular mycorrhizal fungi.

### SUMMARY OF THE DISCLOSURE

The purpose of the present disclosure is to provide the application of phosphate starvation response factor PHR2 in plant and arbuscular mycorrhizal symbiosis, and the application in improving phosphorus nutrition.

In the first aspect, the disclosure provides a method for regulating symbiosis between Gramineous plants and arbuscular mycorrhizal fungi or regulating the inhibitory effect of sufficient phosphate (high phosphate) on mycorrhizal symbiosis, wherein the method comprises: regulating the expression or activity of phosphate starvation response factor PHR in Gramineous plants.

In a preferred embodiment, the method comprises: up-regulating the expression or activity of phosphate starvation response factor PHR in Gramineous plants, thereby promoting the symbiosis of plants and arbuscular mycorrhizal fungi, or antagonizing the inhibitory effect of sufficient phosphate on mycorrhizal symbiosis; preferably, the up-regulation of the expression or activity of phosphate starvation response factor PHR in plants comprises: transferring the encoding sequence of phosphate starvation response factor PHR into plants; or, modifying the promoter region of PHR downstream target genes, adding a P1BS element (preferably, there are at least one, for embodiment two, three, four, five or six P1BS elements in the promoter), or regulating by an up-regulator that interacts with the phosphate starvation regulator PHR, thereby increasing the expression or activity of the phosphate starvation regulator PHR; preferably, the downstream target genes comprise: mycorrhizal symbiosis-specific transcription factor RAM1, mycorrhizal symbiosis-specific transcription factor WRI5A, mycorrhizal symbiosis-specific phosphate transporter PT11 or ammonium transporter AMT3;1.

In another preferred embodiment, the PHR encoding sequence transferred into plants can be transferred into plant cells, tissues or organs; preferably, such as transferred into root tissues.

In another preferred embodiment, the method comprises: down-regulating the expression or activity of phosphate starvation response factor PHR in Gramineous plants, thereby inhibiting the symbiosis of plants and arbuscular mycorrhizal fungi.

In another preferred embodiment, the method comprises: knocking out or silencing the encoding sequence of phosphate starvation response factor PHR, or inhibiting the activity of phosphate starvation response factor PHR, or modifying the promoter region of PHR downstream target genes, reducing P1BS elements (such as deleting P1BS elements); preferably, it comprises: knocking out the encoding sequence of phosphate starvation response factor PHR by CRISPR editing system; knocking out the encoding sequence of phosphate starvation response factor PHR by homologous recombination; silencing the encoding sequence of phosphate starvation response factor PHR by a interfering molecule that specifically interfering the expression; or mutating phosphate starvation response factor PHR by loss-of-function mutation; preferably, the downstream target genes comprise: mycorrhizal symbiosis-specific transcription factor RAM1, mycorrhizal symbiosis-specific transcription factor WRI5A, mycorrhizal symbiosis-specific phosphate transporter PT11 or ammonium transporter AMT3;1.

In another preferred embodiment, in the method, the regulation of symbiosis between Gramineous plants and arbuscular mycorrhizal fungi or the regulation of inhibitory effect of sufficient phosphate on mycorrhizal symbiosis comprises: promoting the efficient acquisition of phosphate in plants; preferably comprising: up-regulating PHR2 under phosphate-deficient (low phosphate) conditions, promoting symbiosis between plants and arbuscular mycorrhizal fungi, and increasing phosphate acquisition; or, under phosphate-sufficient conditions, regulating the plants' own pathways for phosphate acquisition and the symbiosis between plants and arbuscular mycorrhizal fungi to achieve efficient acquisition of phosphate.

In another preferred embodiment, the phosphate-deficient conditions are at an amount of phosphate supply with: 0-50 uM, preferably 0-20 uM, such as 2, 3, 5, 10 or 15 uM.

In another preferred embodiment, the phosphate-sufficient conditions are at an amount of phosphate supply with: 100-400 uM, preferably 150-300 uM, such as 180, 200, 250 or 300 uM.

In another preferred embodiment, said PHR is originates from or said Gramineous plants comprise (but not limited to): rice, maize, wheat, broomcorn, grain, corn, sorghum, millet, barley, rye, oats, Brachypodium distachyon.

In another preferred embodiment, the PHR has a regulating function on downstream target genes, wherein the downstream target genes comprise: mycorrhizal symbiosis-specific transcription factor RAM1, mycorrhizal symbiosis-specific transcription factor WRI5A, mycorrhizal symbiosis-specific phosphate transporter PT11 or ammonium transporter AMT3;1; preferably, the PHR exerts the regulating function by binding to the promoter region of downstream target genes; more preferably, the PHR exerts the regulating function by binding to the P1BS element of downstream target genes; preferably, the regulating function is a function of promoting the symbiosis of plants and arbuscular mycorrhizal fungi, or antagonizing the inhibitory effect of phosphate-sufficient on mycorrhizal symbiosis.

In another preferred embodiment, the downstream target gene regulated by PHR is OsPT11, with 3 P1BS elements in the promoter region: −219/226 bp, −515/522 bp and −1203/1210 bp.

In another preferred embodiment, the phosphate starvation response factor PHR is PHR1, PHR2, PHR3; preferably, it is PHR2; more preferably, it comprises: (a) a polypeptide having the amino acid sequence of SEQ ID NO: 2;

(b) a polypeptide derived from (a) with the function of polypeptide (a) by substitution, deletion or addition of one or several (such as 1-30 or 1-20; preferably 1-10; more preferably 1-5, 1-3 or 1-2) amino acid residues in the amino acid sequence of SEQ ID NO: 2; (c) a polypeptide having more than 50% (preferably more than 60%, more than 65%, more than 70%, more than 75%, more than 80% or more than 85%; more preferably more than 90%; more preferably more than 95%; such as more than 98% or more than 99%) sequence identity to the amino acid sequence in (a) and the function of polypeptide (a); or (d) a fragment of SEQ ID NO: 2 with the function of polypeptide (a).

In another aspect, the disclosure provides a use of phosphate starvation response factor PHR or an encoded gene thereof, or the regulators thereof, for regulating the symbiosis between Gramineous plants and arbuscular mycorrhizal fungi or regulating the inhibitory effect of sufficient phosphate on mycorrhizal symbiosis.

In a preferred embodiment, the regulator is an up-regulator and the phosphate starvation response factor PHR or the up-regulator thereof promotes symbiosis between plants and arbuscular mycorrhizal fungi, or antagonizes the inhibitory effect of sufficient phosphate on mycorrhizal symbiosis; preferably, the up-regulator comprises (but not limited to): an expression cassette or an expression construct (including expression vector) that over-expresses the phosphate starvation regulator PHR; or interacts with the phosphate starvation regulator PHR to increase its expression or activity.

In another preferred embodiment, the regulator is a down-regulator that inhibits symbiosis between plants and arbuscular mycorrhizal fungi; preferably, the down-regulator comprises (but not limited to): an agent knocking out or silencing the encoding sequence of phosphate starvation response factor PHR, an agent inhibiting the activity of phosphate starvation response factor PHR; preferably, the down-regulator comprises: a gene-editing agent, a homologous recombinant agent or an agent for site-directed mutagenesis targeting the encoding sequence of phosphate starvation response factor PHR, the agent brings a loss-of-function mutation to the phosphate starvation response factor PHR; or a interfering molecule that specifically interfering the expression of the encoding sequence of phosphate starvation response factor PHR.

In another aspect, the disclosure provides a use of phosphate starvation response factor PHR or an encoded gene thereof, as a molecular marker for the identification of the symbiosis of Gramineous plants and arbuscular mycorrhizal fungi.

In a preferred embodiment, if the expression of phosphate starvation response factor PHR detected in the tissue of Gramineous plant is higher than a certain value, then relatively speaking, the symbiotic ability of Gramineous plant and arbuscular mycorrhizal fungi is strong; If the expression of phosphate starvation response factor PHR in the tissue of Gramineous plant is lower than a certain value, the symbiotic ability of Gramineous plant and arbuscular mycorrhizal fungi is relatively weak. Wherein, unless otherwise specified, the “certain value” refers to the average value of the expression level of the corresponding phosphate starvation response factor PHR in Gramineous plants.

In another preferred embodiment, the high expression (or expression level is high) or high activity (or activity level is high) refers to a statistically significant increase, such as 10%, 20%, 40%, 60%, 80%, 90% or more, in expression or activity compared with the average expression or activity of the same or same species of plants.

In another preferred embodiment, the low expression (or expression level is low) or low activity (or activity level is low) refers to a statistically significant decrease, such as 10%, 20%, 40%, 60%, 80%, 90% or less, in expression or activity compared with the average expression or activity of the same or same species of plants.

In another preferred embodiment, the “strong symbiotic ability” refers to a statistically significant increase, such as 10%, 20%, 40%, 60%, 80%, 90% or more, compared with the average symbiotic ability of the same or same species of plants and the fungi.

In another preferred embodiment, the “weak symbiotic ability” refers to a statistically significant decrease, such as 10%, 20%, 40%, 60%, 80%, 90% or less, compared with the average symbiotic ability of the same or same species of plants and the fungi.

In another aspect, the present disclosure provides a method for screening substances (potential substances) that regulate the symbiosis of Gramineous plants and arbuscular mycorrhizal fungi or regulate the inhibitory effect of sufficient phosphate on mycorrhizal symbiosis, comprising: (1) Adding candidate substance to the system expressing phosphate starvation response factor PHR; (2) Detecting the system to observe the expression or activity of phosphate starvation response factor PHR in the system; if the expression or activity increases (significantly increases, such as increases 10%, 20%, 40%, 60%, 80%, 90% or more), it indicates that the substance can be used for promoting the symbiosis between Gramineous plants and arbuscular mycorrhizal fungi or antagonizing (or reversing) the inhibitory effect of sufficient phosphate on mycorrhizal symbiosis; if the expression or activity decreases (significantly decreases, such as decreases 10%, 20%, 40%, 60%, 80%, 90% or less), it indicates that the substance can be used for inhibiting the symbiosis between Gramineous plants and arbuscular mycorrhizal fungi.

In a preferred embodiment, the system also expresses downstream target genes of PHR, the downstream target genes comprise: mycorrhizal symbiosis-specific transcription factor RAM1, mycorrhizal symbiosis-specific transcription factor WRI5A, mycorrhizal symbiosis-specific phosphate transporter PT11 or ammonium transporter AMT3;1.

In another preferred embodiment, the method also comprises: observing the binding of phosphate starvation response factor PHR with the downstream target genes, preferably observing the binding of PHR with the promoter region of downstream target genes, more preferably observing the binding of PHR with the promoter element P1BS of downstream target genes; if the binding is enhanced by the substance, it indicates that the substance can be used for promoting the symbiosis between Gramineous plants and arbuscular mycorrhizal fungi; if the binding is weakened by the substance, it indicates that the substance can be used for inhibiting the symbiosis between Gramineous plants and arbuscular mycorrhizal fungi.

In another preferred embodiment, the method also comprises: setting a control group without adding the candidate substance in order to more easily observe the changes of the expression or activity of phosphate starvation response factor PHR between the testing group and the control group.

In another preferred embodiment, the candidate substances comprise (but are not limited to): regulators (such as up-regulators, small-molecule compounds, gene-editing constructs, etc.) designed for phosphate starvation response factor PHR or an encoded gene thereof, or upstream or downstream proteins or genes.

In another aspect, the present disclosure provides a method for identifying mycorrhizal-associated genes regulated by PHR2, comprising analyzing promoters of mycorrhizal-associated genes; wherein, if there is a cis-acting element P1BS, it indicates that the gene can (or potentially can) be directly regulated by PHR2 in mycorrhizal symbiosis.

Other aspects of the present disclosure will be apparent to those skilled in the art based on the disclosure herein.

### DESCRIPTION OF FIGURES

FIG. 1. (A) Expression levels of OsPHR1/2/3 relative to the reference gene Cyclophilin2 in wild-type roots inoculated with (+AM) or without (−AM) mycorrhizal fungi. Error bars represent the standard deviation of three replicates. (B) Schematic representation of OsPHR2 genome with inserted positions of T-DNA and T″ base in osphr2-1 and osphr2-2 mutants. A black solid line indicates introns and 5′/3′ transcribed untranslated regions. Black squares indicate exons. A triangle above the OsPHR2 genome indicates the insertion position of T-DNA. An arrow indicates the location of primer used for mutant identification. Below the OsPHR2 genome is the editing mutant of osphr2-2. “T” in red is the inserted base. “TGA” is a position for premature translation termination. (C) PCR identification of osphr2-1 homozygous mutants. (D)Expression levels of OsPHR2 relative to the reference gene Cyclophilin2 in osphr2-1 and osphr2-2 mutant roots. Error bars represent the standard deviation of three replicates. Asterisks indicate significant differences by t-test compared with the wild type (* P<0.05; ** P<0.01).

FIG. 2. (A) Rate of arbuscular mycorrhizal colonization in wild-type, osphr2-1 and osphr2-2 plants. (B) Statistical results of arbuscule size in wild-type and osphr2-1 plants. Images of arbuscules in osphr2-1 and wild-type plants were photographed under a microscope, with the length of arbuscules measured by Image J. Arbuscule development was measured by the proportion of arbuscules in different sizes. (C) Concentrations of phosphorus in the aerial parts of wild-type, Osphr2-1 and Osphr2-2 plants six weeks after inoculation with mycorrhizal fungi. DW, Dry Weight. (D) Images reveal the arbuscule morphology in wild-type, osphr2-1 and osphr2-2 mutants. Black rectangles in the images are arbuscules stained by ink. In Figure (A) and (C), asterisks indicate significant differences by t-test compared with the wild type (* P<0.05; ** P<0.01).

FIG. 3. (A) The expression of OsPHR2-FLAG protein in OsPHR2 OE2 plants detected by Western Blot. (B) Expression levels of OsPHR2 relative to the reference gene Cyclophilin2 in OsPHR2 OE1 and OE2. Error bars represent the standard deviation of three replicates. (C) Rates of arbuscular mycorrhizal colonization in wild type, PHR2 OE1 and OE2. In Figure (B) and (C), asterisks indicate significant differences by t-test compared with the wild type (* P<0.05; ** P <0.01).

FIG. 4. (A) Rates of mycorrhizal colonization in wild type, osphr2-1 and osphr1/2-1/3 mutants. Different letters (a/b/c) indicate significant differences (ANOVA, Duncan's multiple range tests; P<0.05). (B) Rates of mycorrhizal colonization in wild type, OsPHR1 OE and OsPHR3 OE. Asterisks indicate significant differences compared with the wild type by t-test (* P<0.05).

FIG. 5. Analysis of promoter shows that the promoters of OsPHR1/2/3 target genes contain P1BS elements. There are 2, 3, 3 and 2 P1BS elements in the promoters of OsRAM1, OsWRI5A, OsPT11 and OsAMT3; 1, respectively (short bars indicate P1BS elements).

FIG. 6. EMSA assay in vitro showing specific binding of OsPHR2 to the promoters of OsRAM1(A), OsWRI5A(B), OsAMT3; 1(C) and OsPT11(D), respectively. The first 100 bp and the last 100 bp of the P1BS element on the target gene promoter, plus a total of 208 bp fragment of the P1BS element, were used as an EMSA probe. The two terminals of the probe were labeled with CY5 by PCR amplification for the EMSA assay. The CY5-unlabeled fragment was used as cold probes with 10-fold, 50-fold, 100-fold amounts for competition experiments. Arrows indicate protein-DNA complexes.

FIG. 7. Assay of transcriptional activation in tobacco shows that OsPHR2 can activate the expression of reporter genes driven by downstream target genes, with the fluorescence signal intensity shown in the figure.

FIG. 8. (A) Schematic diagram of the construction of ProPT11: GUS with different P1BS deletions. The yellow-green rectangles represent the 2,600 bp OsPT11 promoter, and the dark green squares represent the P1BS element. (B) Representative images of GUS staining in roots of PT11-1, PT11-2, PT11-3, PT11-4 and PT11-5 plants inoculated with mycorrhizal fungi. Arbuscular mycorrhizal fungi of PT11-5 were also stained by WGA. Cells within the dashed line are the cells with arbuscules.

FIG. 9. Statistics of mycorrhizal colonization rates under phosphate-sufficient and phosphate-deficient conditions in wild type (NIP) and OsPHR2 OE plants. After 3 weeks of inoculation with arbuscular mycorrhizal fungi, the plants were respectively applied with rice nutrient solution containing 0 uM (deficient phosphate) or 200 uM (sufficient phosphate) KH₂PO₄ and the mycorrhizal symbiosis was counted 6 weeks after inoculation. Different letters (a/b/c) indicate significant differences (ANOVA, Duncan's multiple range tests; P<0.05).

FIG. 10. (A) Evolutionary analysis of PHR genes in different species. The evolutionary tree was constructed using maximum likelihood algorithm in MEGA7 with 1,000 bootstrap repetitions. (B) Growth phenotypes of maize Zmphr1/2 double mutants compared to wild-type B73.

### DETAILED DESCRIPTION

After in-depth analyses, the present disclosure revealed an application of phosphate starvation response factor PHR2 in plant and arbuscular mycorrhizal symbiosis and a method for increasing the rate of mycorrhizal symbiosis and phosphate acquisition in plants, so as to finally achieve the purpose of increasing the yield of plants. The inventors also discovered in-vivo mechanism of the phosphate starvation response factor PHR2 in plants that PHR2 binds to the specific position in the promoter of downstream target genes. Therefore, based on the mechanism, it can be used to screen for molecules that regulate mycorrhizal symbiosis or phosphate acquisition by the mechanism.

In one aspect, the present disclosure provides a method for improving arbuscular mycorrhizal colonization in plants, comprising enhancing the gene expression or protein activity of PHR; in the present disclosure, the PHR comprises its homologous gene.

In another aspect, the present disclosure provides a method for identifying mycorrhizal-associated genes regulated by PHR2, comprising analyzing promoters of mycorrhizal-associated genes; wherein, if there is a cis-acting element P1BS, it indicates that the gene can be directly regulated by PHR2 in mycorrhizal symbiosis.

The present disclosure also provides a method for promoting efficient phosphate acquisition in plants, comprising over-expressing PHR2 under phosphate-deficient conditions to improve mycorrhizal symbiosis and phosphate acquisition and coordinating plants' own pathways for phosphate acquisition with mycorrhizal symbiosis to achieve efficient phosphate acquisition under phosphate-sufficient conditions.

Through genetics and molecular biology, the inventors discovered that the phosphate starvation response factor PHR2 directly regulates the expression of arbuscular mycorrhizal specific transcription factors and nutrient transporters in plants and positively regulates the symbiosis of plants and arbuscular mycorrhizal fungi. The inventors also found that the PHR binding element P1BS is necessary for the induced expression of mycorrhizal-associated genes.

### Terms

As used herein, the phosphate starvation response (PHR) gene or polypeptide comprises a PHR gene or polypeptide from rice and a PHR gene or polypeptide that is homologous to a rice-derived gene or polypeptide, with substantially the same structural domains and substantially the same functions.

As used herein, the “symbiosis of Gramineous plants and arbuscular mycorrhizal fungi” is also referred to simply as “mycorrhizal symbiosis” in the present disclosure.

As used herein, the “plants” comprise plants expressing PHR or homologous proteins thereof and plants with PHR or homologous genes thereof in the genome. According to the knowledge in the art, plants expressing PHR or homologs (homologous genes or homologous proteins) thereof have the mechanism as claimed in the present disclosure and can achieve the technical effects as claimed in the present disclosure. The plants can be monocotyledonous plants or dicotyledonous plants. In some preferred embodiments, the plants are crops, preferably cereal crops, and the cereal crops are crops with kernels (grains). In some preferred embodiments, the “cereal crops” can be Gramineous plants; preferably, the Gramineous plants comprise but not limited to: rice, wheat, broomcorn, grain, corn, sorghum, millet, barley, rye, oats, Brachypodium distachyon and so on. The plants can also be legumes and so on.

As used herein, the “aerial part(s)”, also called “aboveground part(s)”, refers to a part of tissue of a plant. When the plant is planted in the ground or cultured in the medium, said tissue is above the ground or the liquid level of medium.

As used herein, the “underground part(s)”, also called “below-ground part(s)”, refers to a part of tissue of a plant. When the plant is planted in the ground or cultured in the medium, said tissue is below the ground or the liquid level of medium.

As used herein, the terms “up-regulate”, “increase”, “improve”, “ameliorate”, “promote”, “enhance” are interchangeable with each other. In the sense of application, it means that when compared with the controls such as “control plant” or “control gene” or “control protein” as defined herein, there is at least 2%, 3%, 4%, 5%, 6%, 7%, 8%, 9% or 10%, preferably at least 15% or 20%, more preferably 25%, 30%, 50%, 80%, 100% or more significant increase.

As used herein, the terms “down-regulate”, “decrease”, “reduce”, “inhibit”, “attenuate”, “block” are interchangeable with each other. In the sense of application, it means that when compared with the controls such as “control plant” or “control gene” or “control protein” as defined herein, there is at least 2%, 3%, 4%, 5%, 6%, 7%, 8%, 9% or 10%, preferably at least 15% or 20%, more preferably 25%, 30%, 50%, 80%, 100% or more significant decrease.

With regard to “control plants”, selection of appropriate control plants is a routine part of experimental design and can comprise corresponding wild-type plants or corresponding transgenic plants without the gene of interest. The control plants are generally the same species or even the same or the same species as the plants to be evaluated. The control plants can also be plants that have lost the transgenic genes by isolation. As used herein, the control plants not only refer to integral plants, but also parts of the plants, including seeds and seed parts.

As used in the present disclosure, the high expression or high activity refers to a statistically significant increase, such as 10%, 20%, 40%, 60%, 80%, 90% or more, in expression or activity compared with the average expression or activity of the same or same species of plants.

As used in the present disclosure, the low expression or low activity refers to a statistically significant decrease, such as 10%, 20%, 40%, 60%, 80%, 90% or less, in expression or activity compared with the average expression or activity of the same or same species of plants.

As used herein, the “promoter” or “promoter region (domain)” refers to a nucleic acid sequence, usually at the upstream (5′ terminal) of the coding sequence of a gene of interest, capable of directing the transcription of the nucleic acid sequence into mRNA. Generally, a promoter or promoter region provides a recognition site for RNA polymerase and other factors necessary for the proper initiation of transcription. As used herein, the promoter or promoter region comprises promoter variants obtained by insertion or deletion of regulatory regions, random or site-directed mutagenesis, and so on.

### Genes and Plants

As used herein, unless otherwise specified, the PHR2 refers to a polypeptide with the sequence of SEQ ID NO: 2 or encoding genes thereof and also comprises variants of the sequence with same functions as the PHR2 polypeptide. The coding gene can be gDNA or cDNA and can also comprise a promoter. For example, the cDNA has the nucleotide sequence shown in SEQ ID NO:1. Sequences of the encoding genes also comprise degenerate sequences from those provided by the present disclosure.

In the present disclosure, the PHR polypeptides also comprise their fragments, derivatives and analogs. As used herein, the term “fragment”, “derivative” or “analog” refers to a protein fragment that essentially maintains the functions or activities of the polypeptides, and may be a protein (i) substituted by one or more conservative or non-conservative amino acid residues (preferably conservative amino acid residues), and such substituted amino acid residues may or may not be encoded by the genetic code, or (ii) with a substitution group in one or more amino acid residues, or (iii) formed by an additional amino acid sequence fused to the protein sequence, and so on. According to the teaching herein, these fragments, derivatives, and analogues belong to the common knowledge to those skilled in the art. Biologically active fragments of the PHR polypeptides can all be applied to the present disclosure.

In the present disclosure, the term “PHR polypeptide” refers to a protein with the sequence of SEQ ID NO: 2 and biological activities of PHR polypeptide. The term also comprises variants of the sequence of SEQ ID NO: 1 with the same function as the PHR polypeptide. The variants may include (but are not limited to): deletion, insertion and/or substitution of one or more (usually 1-50, preferably 1-30, more preferably 1-20, most preferably 1-10, even more preferably 1-8, 1-5) amino acids, and addition or deletion of one or several (usually within 20, preferably within 10, more preferably within 5) amino acids at the C-terminal and/or N-terminal. For example, in the art, substitution with amino acids of approaching or similar property generally does not alter the function of a protein. For another example, the addition of one or several amino acids to C-terminal and/or N-terminal also generally does not alter the function of a protein.

It should be understood that although the PHR gene of the present disclosure are preferably obtained from Gramineous plants, especially rice, but other genes or its degenerate forms obtained from other plants that are highly homologous to the PHR gene of rice (with more than such as 50%, 60%, especially such as 70%, 80%, 85%, 90%, 95%, or even 98% sequence identity) are also within the scope of the present disclosure. Methods and tools for aligning sequence identity are also well known in the art, such as BLAST. As an example, homologue of the PHR gene erived from Triticum aestivum, or Zmphr1/2 derived from Zea mays, but not limited thereto.

Vectors comprising the coding sequences, as well as host cells genetically engineered with the coding sequences of the vectors or polypeptides, are also included in the present disclosure. Methods known to those skilled in the art can be used to construct suitable expression vectors.

Host cells are usually plant cells. For transforming plants, methods such as Agrobacterium transformation or biolistic transformation can generally be used, such as leaf disk method, rice immature embryo transformation method, and so on; preferably Agrobacterium transformation. Transformed plant cells, tissues or organs can be regenerated into plants using conventional methods to obtain plants with altered traits relative to the wild type.

### Cis-Elements and Applications Thereof

During the detailed analyses of the promoters of PHR downstream target genes, the inventors found that when the P1BS was absent on the promoters of PHR downstream target genes (including mycorrhizal-specific promoters and nutrient transporters), the expression of genes would be severely attenuated in mycorrhizal symbiosis, indicating that the P1BS element is crucial for the induction of mycorrhizal symbiotic genes.

Based on this finding of the inventors, the cis-elements can be used as molecular markers to identify downstream target genes of PHR in mycorrhizal symbiosis.

In addition, it can be used for screening substances (potential substances) that regulate the symbiosis of Gramineous plants and arbuscular mycorrhizal fungi or regulate the inhibitory effect of sufficient phosphate on mycorrhizal symbiosis, comprising: (1) Adding candidate substance to the system expressing phosphate starvation response factor PHR; (2) Detecting the system to observe the expression or activity of phosphate starvation response factor PHR in the system; if the expression or activity increases, it indicates that the substance can be used for promoting the symbiosis between Gramineous plants and arbuscular mycorrhizal fungi or antagonizing (reversing) the inhibitory effect of sufficient phosphate on mycorrhizal symbiosis; if the expression or activity decreases, it indicates that the substance can be used for inhibiting the symbiosis between Gramineous plants and arbuscular mycorrhizal fungi.

In preferred embodiments, the system also expresses downstream target genes of PHR, the downstream target genes comprise: mycorrhizal symbiosis-specific transcription factor RAM1, mycorrhizal symbiosis-specific transcription factor WRI5A, mycorrhizal symbiosis-specific phosphate transporter PT11 or ammonium transporter AMT3;1; the method also comprises: observing the binding of phosphate starvation response factor PHR with the downstream target genes, preferably observing the binding of PHR with the promoter region of downstream target genes, more preferably observing the binding of PHR with the promoter element P1BS of downstream target genes; if the binding is enhanced by the substance, it indicates that the substance can be used for promoting the symbiosis between Gramineous plants and arbuscular mycorrhizal fungi; if the binding is weakened by the substance, it indicates that the substance can be used for inhibiting the symbiosis between Gramineous plants and arbuscular mycorrhizal fungi.

The above method is helpful for those skilled in the art to further obtain some useful regulatory molecules for regulating the symbiosis of Gramineous plants and arbuscular mycorrhizal fungi.

The methods for screening substances acting on a protein or gene or its specific region as a target are well known to those skilled in the art, and these methods can be used in the present disclosure. The candidate substances can be selected from: peptides, polymeric peptides, peptidomimetics, non-peptide compounds, carbohydrates, lipids, antibodies or antibody fragments, ligands, small organic molecules, small inorganic molecules, nucleic acid sequences, and the like. Based on the type of substances to be screened, it is clear to those skilled in the art how to select a suitable screening method.

### Applications in the Improvement of Plant Traits

Based on new findings of the inventors, the present disclosure provides a method for improving phosphate acquisition in plants, the method comprises: improving the expression or activity of PHR in plants. Wherein, the improved traits comprise selected from the group consisting of: increasing the expression of mycorrhizal symbiotic genes and increasing the rate of arbuscular mycorrhizal symbiosis.

The symbiotic process of arbuscular mycorrhizal fungi with plants is strictly regulated at the transcriptional level, with many transcription factors in plants involved, including mycorrhizal-specific transcription factors RAM1 and WRI5A. The two factors are involved in regulating fatty acid synthesis and nutrient exchange in mycorrhizal symbiosis, respectively. Phosphate transporter PT11 and ammonium transporter AMT3;1 specifically induced by mycorrhizal symbiosis play an important role in nutrient exchange in mycorrhizal symbiosis. Researches of the present inventors found that there are P1BS elements on the promoters of RAM1. WRI5A, PT11 and AMT3;1. Further experiments showed that PHR2 is indeed involved in regulating the expression of RAM1, WRI5A, PT11 and AMT3;1. These findings of the present disclosure have not been previously studied in the art.

It should be understood that according to the experimental data and regulatory mechanisms provided by the present disclosure, various methods well known to those skilled in the art can be used to regulate the expression of PHR or the downstream target genes RAM1, WRI5A, PT11 and AMT3;1, and these methods are all included in the present disclosure.

In the present disclosure, substances that increase the expression or activity of PHR in plants include agonists, activators and up-regulators. The “up-regulation”, “improvement” or “promotion” includes “up-regulation”, “promotion” of protein activities or “up-regulation”. “improvement” and “promotion” of protein expressions. Any substance that can increase the activity of PHR protein, increase the stability of PHR gene or the encoded protein thereof, up-regulate the expression of PHR gene and increase the effective time of PHR protein can be used in the present disclosure as useful substances for up-regulating PHR genes or the encoded proteins thereof. They can be chemical compounds, small chemical molecules, biomolecules. The biomolecules can be nucleic acids (including DNA, RNA) or proteins.

As another embodiment of the present disclosure, there is also provided a method for up-regulating the expression of PHR genes or encoded proteins thereof in plants, wherein the method comprising: transferring PHR genes, constructs or vectors of the encoding protein thereof into the plants.

In other regulating methods, it also includes down-regulation of the expression or activity of PHR. In the present disclosure, the down-regulators of PHR protein or encoded gene thereof refer to any substance that can decrease the activity of PHR protein, decrease the stability of PHR gene or the encoded protein thereof, down-regulate the expression of PHR protein, decrease the effective time of PHR protein, inhibit the transcription and translation of PHR gene, and reduce the level of phosphorylation/activation of proteins. These substances can be used in the present disclosure as useful substances for down-regulating PHR proteins. They can be chemical compounds, small chemical molecules, biomolecules. The biomolecules can be nucleic acids (including DNA, RNA) or proteins. For example, the down-regulators are: interfering RNAs or antisense nucleotides that specifically interfering the expression of PHR protein or other genes in the pathway; or a gene-editing agent specifically targeting the PHR gene.

As a preferred embodiment, the present disclosure provides a method for down-regulating PHR protein in plants, comprising targeted mutation, gene editing or gene recombination of PHR protein for down-regulation. As a more specific embodiment, by any of the above methods, the PHR protein lost functions any more when converted into the mutant thereof. As a more specific embodiment, the CRISPR/Cas9 system is used for gene editing. Appropriate sgRNA target sites will lead to higher gene editing efficiency. Therefore, before proceeding with gene editing, suitable target sites can be designed and found. After designing specific target sites, in vitro screening for cell activity is also required to obtain effective target sites for subsequent experiments. Preferred gene editing reagents are provided in the examples of the present disclosure.

### The Main Advantages of the Present Disclosure are:

The inventors have deeply studied the mechanisms of PHR in arbuscular mycorrhizal symbiosis and found that key transcription factors of phosphate starvation response factor PHR2 in plants directly regulate mycorrhizal symbiosis by directly regulating the expression of mycorrhizal-related genes including transcription factors, which is of great significance for highly effective phosphate acquisition in plants.

The inventors provides a method for improving plant-arbuscular mycorrhizal symbiosis, thereby providing a feasible method for phosphate acquisition in plants and an effective tool for plant breeding and screening.

The disclosure if further illustrated by the specific examples described below. It should be understood that these examples are merely illustrative, and do not limit the scope of the present disclosure. The experimental methods without specifying the specific conditions in the following examples generally used the conventional conditions, such as those described in J. Sambrook, Molecular Cloning: A Laboratory Manual (3rd ed. Science Press, 2002) or followed the manufacturer's recommendation. Unless otherwise defined, all professional and scientific terms used herein have the same meanings as familiar to those skilled in the art. In addition, any methods and materials similar or equivalent to those described can be used in the present disclosure. Methods and materials for preferred embodiments described herein are provided for illustrative purposes only.

### I. Materials and Methods

### 1. Experimental Materials

### 1.1. Plant Materials

In the present disclosure, all wild-type rice materials used as a control are obtained from Nipponbare (Oryza sativa ssp. Japonica cv. Nipponbare). The mutant osphr2-1 used in the present disclosure is a T-DNA inserted mutant based on the background of Nipponbare. The mutant osphr2-2 and oswri5a/b were obtained by DNA editing of OsPHR2, OsWRI5A and OsWRI5B genomes in Nipponbare with CRISPR/CAS9 gene editing system.

The tobacco used in the present disclosure is Nicotiana benthamiana.

### 1.2. Strains and Vectors

Escherichia coli for constructing the vector: DH5a, CCDB3.1.

Agrobacterium tumefaciens: GV3101, EHA105.

Entry Vector: pCR-Blunt, pDONR207 and pENTR.

CRISPR/CAS9 gene vector: The target sequence of OsPHR2, CAGTCCAGTACCGGGTCTGTTGG (SEQ ID NO: 3), was synthesized and connected to the intermediate vector pOs-sgRNA, and the proOsU3-OsPHR2 target sequence-gRNA expression cassette was connected to the vector pH-Ubi-cas9-7 by LR recombination reaction. Wherein proOsU3 is the promoter of OsU3 (X79685.1).

pCAMBIA1301-OsPHR2: obtained by inserting the coding region of OsPHR2 (LOC_Os07g25710) into the multiple cloning sites of pCAMBIA1301.

pCAMBIA1300-pOsPT11-GUS: obtained by inserting the promoter of OsPT11 (LOC_Os01g46860, 2600 bp before ATG) into the multiple cloning sites of modified pCAMBIA1301 (with gene sequence encoding GUS inserted between the multi-cloning sites BamHI and EcoRI).

pMAL-C2x-OsPHR2: obtained by inserting the coding region of OsPHR2 (LOC_Os07g25710) into the multiple cloning sites of pMAL-C2x.

Tobacco transcription activation vector: obtained by inserting the promoters of OsRAM1 (LOC_Os11g31100), WRI5A (LOC_Os06g05340), OsPT11 (LOC_Os01g46860) and OsAMT3;1 (LOC_Os01g65000) into the multiple cloning sites of the luciferase reporter gene vector pLLOOR.

### 2. Experimental Methods

### 2.1 Agrobacterium-Mediated Transformation of Rice

Firstly, immature embryo callus of rice is prepared for the following Agrobacterium-competent preparation and transformation. After culture of Agrobacterium (engineering bacteria), infection and co-culture were performed. The callus after co-cultivation was obtained for resistance screening. After that, resistant callus was induced for differentiation and rooting. Above steps are all carried out under sterile conditions.

2.2 Arbuscular Mycorrhizal Fungi Inoculation and Phosphate Treatments with Different Concentrations

Rice seeds with the chaff removed were surface-sterilized with 75% ethyl alcohol for 1 min and sodium hypochlorite (sodium hypochlorite: water=1:2.5) for 30 min, followed by five washes with sterile ddH₂O. The surface-sterilized seeds were transferred to tubes with solid ½ MS medium, then germinated and grew for two weeks in phytochamber with 12 h light/12 h dark at 28° C./22° C. and 70% humidity.

The sand containing arbuscular mycorrhizal fungal spores was mixed with vermiculite at a ratio of 1:4 and placed in a 5×10 black plug tray. Then the seedlings were transplanted into the plug tray and cultured in artificial green house. When the rice seedlings were inoculated with arbuscular mycorrhizal fungi, small amounts of tap water were daily supplied in the first two weeks. From the third week, nutrient solutions (see Appendix Table 2) were applied every two days. After 6 weeks, the seedlings were dug, with the roots stained and colonization rate counted.

For phosphate treated, 0 uM, 100 uM and 200 uM KH₂PO₄ were added to the nutrient solution. The nutrient solution was applied at the third week after inoculation and samples were taken after 6 weeks to detect mycorrhizal colonization.

### 2.3 Root Staining and Statistics

(1) Rice roots co-cultured with arbuscular mycorrhizal fungi were taken, washed, dried by wipe, and placed in a 2 ml EP tube with a hole at the bottom.
(2) 10% KOH and ink (with 5 ml ink, 5 ml glacial acetic acid and 90 ml of water to 100 ml) were respectively added into a dyeing box and preheated at 95° C.
(3) The rice roots were placed in 10% KOH for 12 min and taken out.
(4) Rinsed by water for three times.
(5) Stained with ink for 6 min, then taken out and put in a 72-hole blue box for decolorization, with water changed frequently.
(6) Observe and count under a stereomicroscope.

The roots stained with ink were placed in a dish with grids (0.5 cm×0.5 cm) and counted under a stereomicroscope.

The ink-stained rice roots were observed under a microscope and the arbuscular structure was photographed at the same magnification. The arbuscules photographed under the microscope was placed in the software MacBiophotonics ImageJ to measure the length of arbuscules. Based on the scale bar in the images, length of arbuscules were counted and then arbuscules that fall at each length region were counted and plotted.

### 2.4 Electrophoretic Mobility Shift Assay (EMSA)

a. Required Reagents:

5×TBE solution (1 L): Tris: 54 g, boric acid: 27.5 g, Na2EDTA.2H₂O: 3.72 g, the pH was automatically 8.3 after preparation.

5×EMSA buffer: Tris-HCl (PH8.0): 100 mM, glycerol: 25%, BSA: 0.2 mg/ml; filtered and sterilized after preparation and frozen at −20° C. for use.

b. Experimental Procedure

(1) Probe preparation: primers are designed, and a universal linker: CY5-AGCCAGTGGCGATAAG (SEQ ID NO: 4) was added in front of the forward primer and the reverse primer. After synthesizing the primers, the probe fragments were amplified from the template. The recovered fragment 1 was amplified for the second time with the universal primer CY5-AGCCAGTGGCGATAAG labeled with CY5 at the 5′ terminal and recovered to obtain fragment 2 (Because CY5 can be decomposed by light, so light is avoided as much as possible when preparing fluorescent probes). Fragment 1 and Fragment 2 were measured for concentration and then diluted to a concentration of 0.08 pmol. Fragment 1 that is not labeled with CY5 is the cold probe for competition experiments, and fragment 2 that is labeled with CY5 is the probe.

(2) Preparation of non-denaturing PAGE gel: Before gel preparation, the mold used for gel preparation must be rinsed to ensure that there is no SDS residue, and a 4% gel was prepared. To prepare a 4% non-denaturing PAGE gel, 700 uL 5×TBE solution, 700 uL 40% acrylamide (29:1), 350 uL 50% glycerol, 7 uL tetramethylethylenediamine (TEMED), 5.3 ml ddH₂O, 35 uL 10% ammonium persulfate (AP) were required. Gelatinize for one to two hours.

(3) Reaction system preparation

Firstly 5× binding buffer was prepared. The system is as follows: Reagents (the amount added in 80 uL): 5×EMSA buffer: 70.8 uL, 1M MgCl₂: 4 uL, 0.5M DTT: 0.8 uL, H₂O: 4.4 uL;

Then a 20 uL reaction system was prepared according to the following table:
5× binding buffer: 4 uL
0.1 M KCl: 3 uL
Salmon sperm DNA (50 ng/uL): 0.2 uL
Purified protein: 1 uL (about 50 ng)
Cy5-labeled probe: 1 uL
H₂O: 10.8 uL

(4) After preparation, the reaction system was placed at room temperature for 30 min.

(5) Pre-electrophoresis was performed in half an hour of the protein-probe reaction: A clean electrophoresis tank (with no SDS residue) was used for pre-electrophoresis. After the non-denatured gel completely solidified, the gel was put into the electrophoresis tank (the electrophoresis tank is placed in an ice-water mixture), with 1×TBE that has been fully pre-cooled in advance added and the comb carefully removed. Pre-electrophoresis at 120V on ice for half an hour.

(6) Electrophoresis: After the reaction, 2.3 uL of 10× loading buffer (250 mM Tris-HCl [PH 7.5], 40% glycerol, 0.2% bromophenol blue) was added to each tube and mixed gently with a pipette for several times. Pre-electrophoresis was stop running. 12 uL of sample was added to the well, electrophoresis at 120V on ice for 90 min in the dark and the electrophoresis was stop running.

(7) Gel scanning: After running, the PAGE gels were scanned using an FUJIFILM FLA 9000plus DAGE at the CY5 channel, with images saved.

2.5 Transient Expression Assay of Nicotiana benthamiana Proteins
a. Required Reagents:

Infiltration buffer: MgCl₂: 10 mM, ethanesulfonic acid solution (pH 5.6): 10 mM, acetosyringone: 150 uM.

b. Experimental Procedure

(1) Transformed bacteria GV3101 stored at −80° ° C. were streaked on the corresponding resistant plates for activation and cultured at 28° C.

(2) A single clone was extracted from the cultured plate in 8 ml of the corresponding resistant LB medium, cultured overnight at 28° C. with constant shaking at 200 rpm.

(3) Centrifugation at 8000 rpm for 8 min, with the supernatant discarded.

(4) The bacteria were washed with infiltration buffer twice and then resuspended in infiltration buffer until OD600 is 1.0.

(5) According to experimental requirements, the resuspensions in different corresponding combinations were mixed with P19 at equal volume (1:1:1), inverted and mixed to a final mixture and placed at 28° C. for 2 hours.

(6) Fully extended leaves of well-grown tobacco were picked and infiltrated with bacterial liquid from the back of leaves into the tissues of leaves by a 1 ml needleless syringe, with marks to distinguish different combinations.

(7) After 48h infiltrated, the Nicotiana benthamiana leaves were cut and sprayed with luciferase substrate evenly on the front of the leaves, and then the luciferase signals were captured using a cold CCD camera in the dark for 5 min.

### 2.6 GUS Staining

(1) Fresh plant tissues were cut into a 10 ml centrifuge tube, with GUS staining buffer added to cover the plant tissues.

(2) A vacuum treated for 20 min.

(3) The samples were placed at 37° C. in dark, with dyeing conditions obsereved every one hour.

(4) After discarding the GUS staining buffer, the staining was terminated by adding 50% ethanol.

(5) Images were taken using a microscope.

### II. EXAMPLE

### Example 1. OsPHR1/2/3 are not Induced by Mycorrhizal Symbiosis

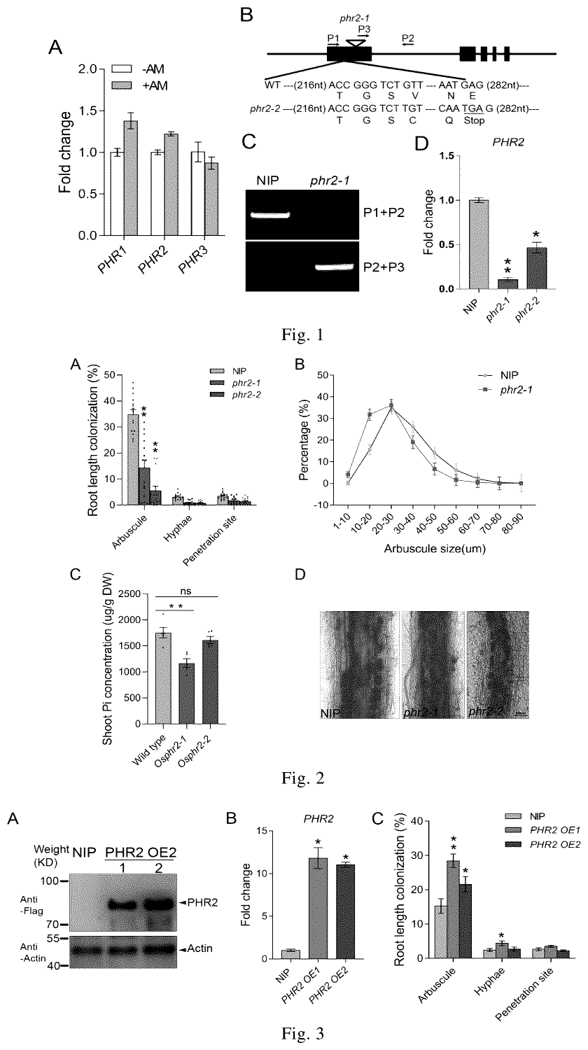

The inventors determined expression levels of OsPHR1/2/3 relative to the reference gene Cyclophilin2 in wild-type roots inoculated with (+AM) or without (−AM) mycorrhizal fungi. It was found that the expression of OsPHR2 and homologous genes OsPHR1 and OsPHR3 were not induced by mycorrhizal symbiosis (FIG. 1A).

Wherein, the nucleotide sequence of OsPHR2 is as follows (SEQ ID NO: 1):
atggagagaataagcaccaatcagctctacaattctggaattccggtgactgtgccatcgcctctgcctgctataccagctaccctggatgaaaacat
tcccaggattccagatgggcagaatgttccgcgggagagagaattgagaagcacacctatgccacctcatcagaatcagagtactgttgctcctcttc
atgggcattttcagtccagtaccgggtctgttgggcctctgcgttcgtcccaggcgataaggttctcttcagtttcaagcaatgagcaatatacaaat
gccaatccttacaattctcaaccgccgagtagtgggagttcttcaacgctcaattatggatcacaatatggaggctttgaaccttccttgactgattt
tccaagagatgctgggccgacgtggtgtcctgatccagttgatggcttgcttggatatacagatgatgtccctgctgggaacaatttgactgaaaaca
gttctattgcagctggtgatgaacttgccaagcaaagtgaatggtggaatgattttatgaattatgactggaaagatattgataacacagcttgtact
gaaactcaaccacaggttggaccagctgcgcaatcatctgtcgcagttcaccaatcagctgcccaacaatcagtttcatctcaatcaggagaaccttc
tgcagttgctataccctcgccctctggtgcctccaatacctccaactccaagacacgaatgagatggactcctgaacttcatgagcgctttgtagatg
ctgtcaatctacttggtggcagtgaaaaagctactcccaagggtgtgttaaagctaatgaaggcagacaatttgaccatttatcatgttaaaagtcac
cttcagaaatacagaacagctcgatacagaccagaattgtctgaaggttcttcagaaaagaaggcagcctcaaaagaggacataccatcaatagatct
gaaaggagggaactttgatctcactgaggcattgcgtctccagttagaactccaaaagaggcttcatgaacagcttgagatccaaagaagtttgcagc
tgagaattgaggagcaagggaagtgccttcagatgatgctcgagcagcagtgcatacctgggacagacaaggcggtggatgcttcaacctcagcagaa
ggaacaaagccatcttctgatcttccagaatcttctgccgtgaaggatgttccagagaacagtcagaacggaatagccaaacaaacagaatcaggtga
cagataa
The nucleotide sequence of OsPHR2 is as follows (SEQ ID NO: 2):
MERISTNQLYNSGIPVTVPSPLPAIPATLDENIPRIPDGQNVPRERELRSTPMPPHQNQSTVAPLHGHFQSST
GSVGPLRSSQAIRFSSVSSNEQYTNANPYNSQPPSSGSSSTLNYGSQYGGFEPSLTDFPRDAGPTWCPDPVDGLLG
YTDDVPAGNNLTENSSIAAGDELAKQSEWWNDFMNYDWKDIDNTACTETQPQVGPAAQSSVAVHQSAAQQSV
SSQSGEPSAVAIPSPSGASNTSNSKTRMRWTPELHERFVDAVNLLGGSEKATPKGVLKLMKADNLTIYHVKSHL
QKYRTARYRPELSEGSSEKKAASKEDIPSIDLKGGNFDLTEALRLQLELQKRLHEQLEIQRSLQLRIEEQGKCLQ
MMLEQQCIPGTDKAVDASTSAEGTKPSSDLPESSAVKDVPENSQNGIAKQTGMRIH

The inventors obtained two mutants of OsPHR2: Osphr2-1 and Osphr2-2. Among them, Osphr2-1 is a T-DNA inserted mutant involved in phosphate starvation response reported by Chen et al. (Chen, J. et al. (2011). Plant Physiology 157, 269-278) (FIG. 1C), and Osphr2-2 is a stably transformed new mutant obtained by CRIPSR/CAS9 in the present disclosure, with a base “T” inserted between 223 and 224 bp and leading to the translation termination prematurely of OsPHR2 protein (FIG. 1B).

Real-time fluorescence-quantitative PCR results showed that expressions of OsPHR2 in both Osphr2-1 and Osphr2-2 mutants were significantly lower than that of the wild type (FIG. 1D).

### Example 2. Rate of Mycorrhizal Colonization Reduced in Osphr2 Mutants

The inventors analyzed rates of arbuscular mycorrhizal colonization in wild-type, osphr2-1 and osphr2-2 plants. The results showed that rates of mycorrhizal colonization in two Osphr2 mutants were significantly lower than those of the wild type (FIG. 2A), indicating that rice-arbuscular mycorrhizal symbiosis was severely affected by loss-of-function of OsPHR2. To better reflect mycorrhizal symbiosis, the inventors measured the size of arbuscules formed in NIP and Osphr2-1 mutants and plotted the results.

The results showed that in Osphr2-1 mutant, the arbuscules were slightly smaller than that of the wild type, with normal arbuscule morphology (FIG. 2B). The arbuscule morphologies in osphr2-1 and osphr2-2 mutants showed in FIG. 2D.

Six weeks after inoculation with mycorrhizal fungi, concentrations of phosphorus in the aerial parts of wild-type, Osphr2-1 and Osphr2-2 plants were measured, wherein the concentration of phosphorus decreased in Osphr2-2 plants compared with the wild-type plants, with the concentration of phosphorus decreased significantly in Osphr2-1 plants (FIG. 2C).

### Example 3. Overexpression of OsPHR2 Increases the Rate of Mycorrhizal Colonization

The inventors further obtained OsPHR2-overexpressed plants. Wherein, OsPHR2 OE1 is driven by 35S promoter, OsPHR2 OE2 is driven by Ubiquitin promoter.

Quantitative results showed that the expression level of OsPHR2 in OsPHR2 OE plants was significantly higher than that in wild type (FIG. 3B). Western blotting showed that OsPHR2 protein accumulated in OsPHR2 OE2 (FIG. 3A).

The inventors used NIP and OsPHR2 OE plants for mycorrhizal colonization and found that six weeks after inoculation with arbuscular mycorrhizal fungi, the rates of mycorrhizal colonization in OsPHR2 OE1 and OsPHR2 OE2 were significantly higher than those of the wild type (FIG. 3C), further indicating that OsPHR2 regulates rice-arbuscular mycorrhizal symbiosis.

### Example 4. OsPHR2, OsPHR1 and OsPHR3 Function Redundantly in Mycorrhizal Symbiosis

The inventors also examined mycorrhizal colonization in OsPHR1-overexpressed and OsPHR3-overexpressed plants and found that the rate of mycorrhizal colonization in OsPHR1-overexpressed plants was also significantly higher than that of the wild type. This data was consistent with the phenotype of OsPHR1-overexpressed plants. Also, the rate of mycorrhizal colonization in OsPHR3-overexpressed plants increased to some extent (FIG. 4B).

The inventors obtained Osphr1/2-1/3 triple mutant by hybridization. By statistically analyzing mycorrhizal phenotypes of the NIP. Osphr2-1 and Osphr1/2-1/3 triple mutant, it was found that after inoculated with arbuscular mycorrhizal fungi for 6 weeks, the rate of mycorrhizal colonization in the Osphr1/2-1/3 triple mutant was further reduced, which is significantly lower than that of the Osphr2-1 mutant, with almost no colonization (FIG. 4A).

Above results suggest that OsPHR1/2/3 are essential for mycorrhizal symbiosis, and these three genes function redundantly in regulating mycorrhizal symbiosis.

### Example 5. Analysis of Promoter Cis-Elements in OsPHR1/2/3 Downstream Target Genes

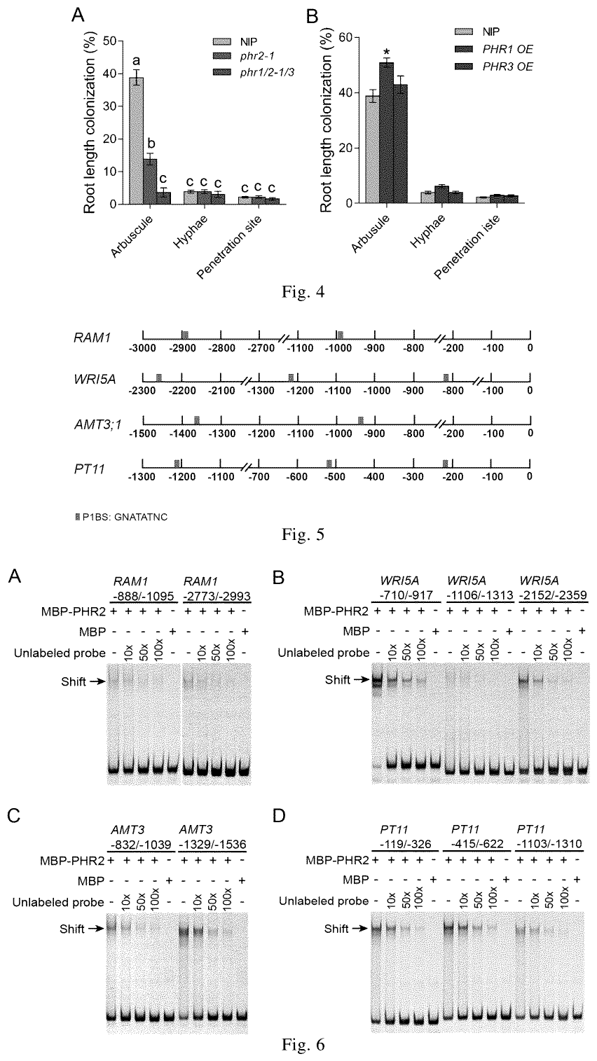

The inventors analyzed mycorrhizal-specific genes in rice and found that there are at least two P1BS elements in promoters of four genes including mycorrhizal-specific transcription factors OsRAM1, OsWRI5A and mycorrhizal symbiosis-specific transporters OsPT11, OsAMT3;1 (2, 3, 3 and 2 P1BS elements on the promoters of OsRAM1, OsWRI5A, OsPT11 and OsAMT3; 1, respectively) (FIG. 5).

Above results suggest that OsPHR1/2/3 directly bind to the promoters of these mycorrhizal symbiosis-specific genes and regulate their expression.

### Example 6. OsPHR2 Directly Binds to Promoters of Downstream Target Genes

The inventors performed an in-vitro electrophoretic mobility shift assay (EMSA), and the results showed that MBP-PHR2 can interact with promoter probes from OsRAM1 (FIG. 6A), OsWRI5A (FIG. 6B), OsAMT3;1 (FIG. 6C) and OsPT11 (FIG. 6D). The corresponding cold probe can compete with the probe to bind MBP-PHR2, indicating the specificity of MBP-PHR2 binding to the probe. Moreover, intensities of MBP-PHR2 binding to probes at different positions on the same gene promoter was significantly different (FIG. 6).

Above results indicated that OsPHR2 protein could bind to the promoters of downstream target genes under in vitro conditions.

### Example 7. OsPHR2 can Activate Downstream Target Genes

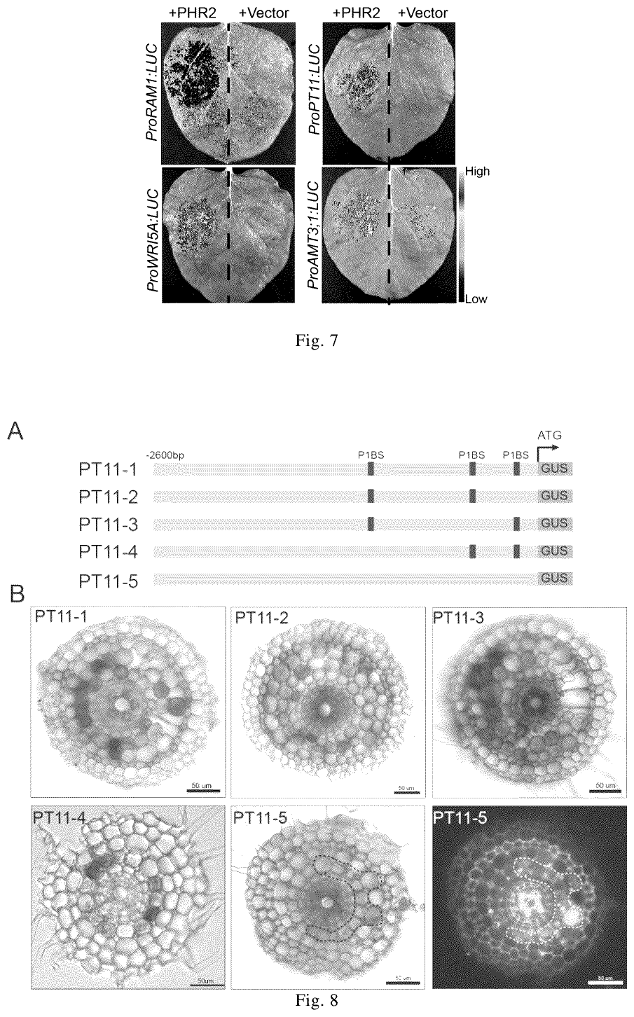

To investigate whether the binding of OsPHR2 to OsRAM1, OsWRI5A, OsPT11 and OsAMT3;1 promoters can activate their expression, the inventors conducted transcriptional activation experiments in tobacco leaves and found that after OsPHR2 protein and luciferase (Promoter: Luciferase) driven by target gene promoters were expressed together in tobacco leaves for 36 hours, OsPHR2 significantly activated the reporter gene expression of luciferase (FIG. 7).

This result suggests that OsPHR2 can activate the expression of downstream target genes for mycorrhizal symbiosis via binding to P1BS elements of the promoters.

### Example 8. P1BS is Essential for Mycorrhizal Induction of OsPT11

To determine whether the P1BS element is essential for induced expression of genes during mycorrhizal symbiosis, the inventors selected the promoter of the phosphate transporter gene OsPT11 in mycorrhizal symbiotic phosphate transport for further analysis.

The inventors found that there are three P1BS elements on the promoter of OsPT11: −219/226 bp, −515/522 bp and −1203/1210 bp (FIG. 8A).

In order to study the effect of P1BS on OsPT11 expression in mycorrhizal symbiosis, the inventors constructed various vectors for promoter expression analysis based on 2,600 bp promoter of OsPT11 with deletion of three P1BS elements respectively and simultaneously (ProPT11: GUS): full-length promoter without P1BS deletion (PT11-1), OsPT11 promoter with P1BS deletion of −219/226 bp (PT11-2), OsPT11 promoter with P1BS deletion of −515/522 bp (PT11-3), OsPT11 promoter with P1BS deletion of −1203/1210 bp (PT11-4) and OsPT11 promoter with P1BS deletion of −219/226 bp, −515/522 bp and −1203/1210 bp (PT11-5) (FIG. 8A). Theses vectors were used for stable rice transformation. Stably-transformed ProPT11:GUS transgenic rice was used for mycorrhizal fungal infection. Six weeks after infection, GUS staining was performed. GUS staining analysis showed that in PT11-1, PT11-2, PT11-3 and PT11-4 transgenic roots, GUS was specifically expressed in cells with arbuscules, indicating that removing a single PBS element of OsPT11 promoter did not affect its expression in mycorrhizal symbiosis. Interestingly, in PT11-5 transgenic roots, although arbuscules were formed in roots of rice, there was no GUS staining (FIG. 8B), indicating that three P1BS elements are essential for OsPT11 expression induced by mycorrhizal fungi in arbuscular cells. It indicated that OsPHR2 regulates the expression of mycorrhizal-associated genes via P1BS elements.

### Example 9. Overexpression of OsPHR2 can Antagonize the Inhibitory Effect of Sufficient Phosphate on Mycorrhizal Symbiosis

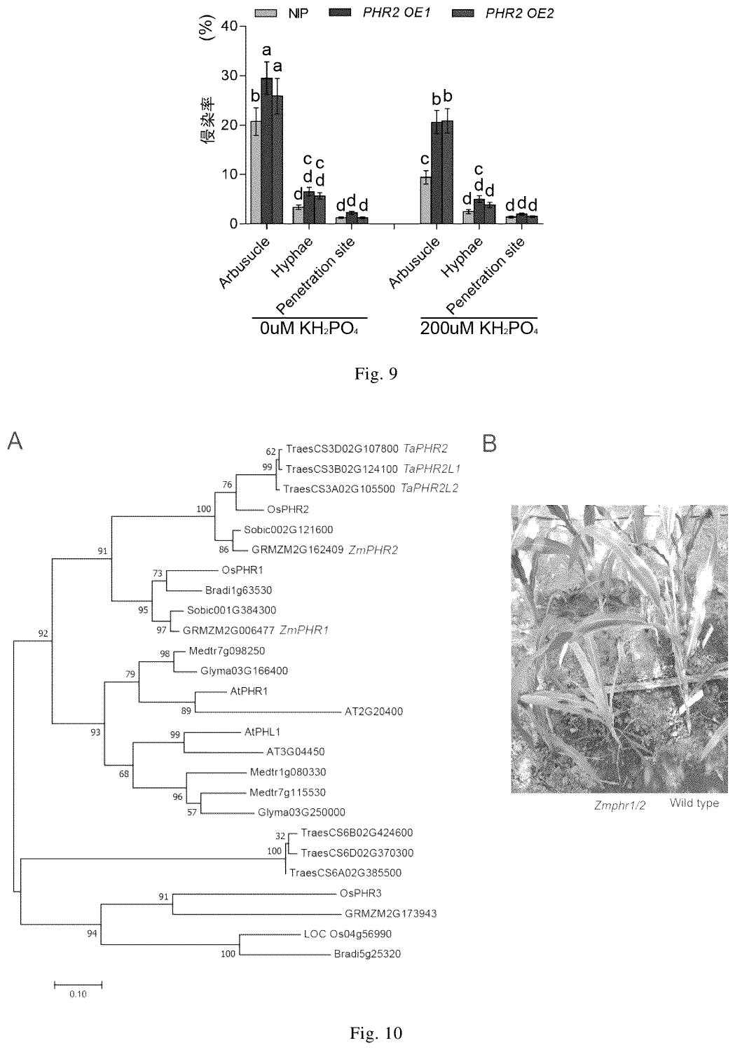

The inventors found that overexpression of OsPHR2 enhanced mycorrhizal symbiosis, and further speculated that OsPHR2 may play an important role in the inhibition of mycorrhizal symbiosis by high phosphate. The inventors detected mycorrhizal symbiosis of wild-type NIP and OsPHR2 OE strains respectively under phosphate-sufficient and phosphate-deficient condition (low phosphate condition) (rice seedlings were supplied by nutrient solution without additional phosphate) and found that the rate of mycorrhizal colonization in OsPHR2 OE strains under phosphate-sufficient condition (high phosphate condition) was significantly higher than that of the wild type (FIG. 9).

More interestingly, the rate of mycorrhizal colonization in OsPHR2 OE strains under phosphate-sufficient condition was comparable to that of the wild type under phosphate-deficient condition, showing insensitivity to phosphate-sufficient (high phosphate) nutrition (FIG. 9). It indicated that OsPHR2 overexpression could antagonize the inhibitory effect of sufficient phosphate on mycorrhizal symbiosis.

### Example 10. Functional Conservation of OsPHR2 in Zea mays and Triticum aestivum

1. Zea mays

The inventors found homologous genes of OsPHR2 in Zea mays through evolutionary analysis and named them ZmPHR1 (GRMZM2G006477) and ZmPHR2 (GRMZM2G162409). The amino acid sequence homology between ZmPHR1 and OsPHR2 was 38.97%; the amino acid sequence homology between ZmPHR2 and OsPHR2 was 65.4%.

A Zmphr1/2 homozygous mutant was obtained by simultaneous knockout of ZmPHR1/2 in Zea mays using Crispr/Cas9. The deletion of 406 bp and 407 bp in ZmPHR1 results in premature termination of protein translation (137aa); the deletion of bases 95-99 of ZmPHR2 results in premature termination of protein translation (89aa) to obtain.

The results showed that Zmphr1/2 mutant plants were stunted compared with the wild type, with the plant height significantly lower than that of the wild type (FIG. 10B).

2. Triticum aestivum

The inventors found a homologous gene of OsPHR2 in Triticum aestivum through evolutionary analysis and named it TaPHR2 (TraesCS3D02G107800). A polynucleotide encoding the gene was inserted into over-expressed vector pCAMBIA1301 and transformed into the Osphr2-1 mutant by the inventors. The results showed that overexpression of TaPHR2 in Triticum aestivum could partially restore the mycorrhizal phenotype of Osphr2-1 mutants, indicating that the function of OsPHR2 is conserved in monocotyledonous plants Triticum aestivum and Zea mays.

### Discussion

In conclusion, through genetics and molecular biology, the inventors discovered that phosphate starvation response factor PHR2 positively regulates rice-arbuscular mycorrhizal symbiosis by directly regulating the expression of mycorrhizal-associated genes.

Overexpression of OsPHR1 and OsPHR2 can significantly increase the efficiency of mycorrhizal symbiosis. Overexpression of OsPHR3 also shows a certain promoting effect, but it is relatively lower than that of OsPHR1 and OsPHR2. This may be because the binding ability of OsPHR3 to P1BS elements is weaker than that of OsPHR1 and OsPHR2, or other regulatory feedback exists in OsPHR3.

The inventors found that OsPHR2 is a core positive regulator in mycorrhizal symbiosis, and OsPHR2 directly regulates the expression of mycorrhizal-associated transcription factors and other genes in mycorrhizal symbiosis. The expression of OsPHR2 was not induced by mycorrhizal symbiosis, indicating that it was mainly regulated at protein level in mycorrhizal symbiosis. At present, yeast two-hybrid results of the present inventors have not showed that OsPHR2 interacts with OsCCAMK and OsCYCLOP. Explanations suggesting that OsPHR2 is involved in mycorrhizal symbiosis are: (1) By phosphorylating OsPHR2 or the interaction of OsPHR2 with other common symbiotic signal components, OsPHR2 is activated by symbiotic signals and regulates the expression of symbiotic genes; (2) OsPHR2 acts as the core of signal network between mycorrhizal symbiosis and phosphate nutrient stress to monitor nutrient status in soil, such as phosphate nutrient concentration. In phosphate-deficient soil, the mycorrhizal symbiotic network is opened for establishing high-efficient mycorrhizal symbiosis under the stimulation of mycorrhizal factors and other signals; while in phosphate-sufficient conditions, the network is in an inactive state for inhibiting mycorrhizal symbiosis.

The inventors demonstrated that P1BS is necessary for the induction of mycorrhizal symbiotic genes by removing the P1BS element of OsPT11 promoter (FIG. 8). EMSA assay showed that OsPHR2 had different binding abilities to P1BS elements at different positions on the promoter (FIG. 6), suggesting that the binding of OsPHR2 to P1BS elements was affected by adjacent sequences of P1BS and the P1BS elements at different positions of the promoter may have certain functional complementarity.

Each reference provided herein is incorporated by reference to the same extent as if each reference was individually incorporated by reference. In addition, it should be understood that based on the above teaching content of the disclosure, those skilled in the art can practice various changes or modifications to the disclosure, and these equivalent forms also fall within the scope of the appended claims.

1. A method for regulating symbiosis between Gramineous plants and arbuscular mycorrhizal fungi or regulating the inhibitory effect of sufficient phosphate on mycorrhizal symbiosis, wherein the method comprises: regulating the expression or activity of phosphate starvation response factor PHR in Gramineous plants.

2. The method of claim 1 comprising: up-regulating the expression or activity of phosphate starvation response factor PHR in Gramineous plants, thereby promoting the symbiosis of plants and arbuscular mycorrhizal fungi, or antagonizing the inhibitory effect of sufficient phosphate on mycorrhizal symbiosis.

3. The method of claim 1, wherein, it comprises: down-regulating the expression or activity of phosphate starvation response factor PHR in Gramineous plants, thereby inhibiting the symbiosis of plants and arbuscular mycorrhizal fungi.

4. The method of claim 3, wherein, it comprises: knocking out or silencing the encoding sequence of phosphate starvation response factor PHR, or inhibiting the activity of phosphate starvation response factor PHR, or modifying the promoter region of PHR downstream target genes, reducing P1BS elements.

5. The method of claim 1, wherein, the regulation of symbiosis between Gramineous plants and arbuscular mycorrhizal fungi or the regulation of inhibitory effect of sufficient phosphate on mycorrhizal symbiosis comprises: promoting the efficient acquisition of phosphate in plants; preferably comprising: up-regulating PHR2 under low-phosphate conditions, promoting symbiosis between plants and arbuscular mycorrhizal fungi, and increasing phosphate uptake; or, under phosphate-sufficient conditions, regulating plants' own pathways for phosphate acquisition and the symbiosis between plants and arbuscular mycorrhizal fungi to achieve efficient acquisition of phosphate.

6. The method of claim 1, wherein, the PHR-derived Gramineous plants or the Gramineous plants comprise: rice, maize, wheat, broomcorn, grain, corn, sorghum, millet, barley, rye, oats, or Brachypodium distachyon.

7. The method of claim 1, wherein, the PHR has a regulating function on downstream target genes, wherein the downstream target genes comprise: mycorrhizal symbiosis-specific transcription factor RAM1, mycorrhizal symbiosis-specific transcription factor WRI5A, mycorrhizal symbiosis-specific phosphate transporter PT11 or ammonium transporter AMT3;1; preferably, the PHR exerts the regulating function by binding to the promoter region of downstream target genes; more preferably, the PHR exerts the regulating function by binding to the P1BS element of downstream target genes; preferably, the regulating function is a function of promoting the symbiosis of plants and arbuscular mycorrhizal fungi, or antagonizing the inhibitory effect of sufficient phosphate on mycorrhizal symbiosis.

8. The method of claim 1, wherein, the phosphate starvation response factor PHR is PHR1, PHR2, or PHR3; preferably, it is PHR2.

9-12. (canceled)

13. A method for screening substances that regulate the symbiosis of Gramineous plants and arbuscular mycorrhizal fungi or regulate the inhibitory effect of sufficient phosphate on mycorrhizal symbiosis, comprising: (1) Adding candidate substance to the system expressing phosphate starvation response factor PHR; (2) Detecting the system to observe the expression or activity of phosphate starvation response factor PHR in the system; if the expression or activity increases, it indicates that the substance can be used for promoting the symbiosis between Gramineous plants and arbuscular mycorrhizal fungi or antagonizing the inhibitory effect of sufficient phosphate on mycorrhizal symbiosis; if the expression or activity decreases, it indicates that the substance can be used for inhibiting the symbiosis between Gramineous plants and arbuscular mycorrhizal fungi.

14. The method of claim 13, wherein, the system also expresses downstream target genes of PHR, the downstream target genes comprise: mycorrhizal symbiosis-specific transcription factor RAM1, mycorrhizal symbiosis-specific transcription factor WRI5A, mycorrhizal symbiosis-specific phosphate transporter PT11 or ammonium transporter AMT3;1;
the method also comprises: observing the binding of phosphate starvation response factor PHR with the downstream target genes, preferably observing the binding of PHR with the promoter region of downstream target genes.

15. A method for identifying mycorrhizal-associated genes regulated by PHR2, comprising analyzing promoters of mycorrhizal-associated genes; wherein, if there is a cis-acting element P1BS, it indicates that the gene can be directly regulated by PHR2 in mycorrhizal symbiosis.

16. The method of claim 2, wherein the up-regulation of the expression or activity of phosphate starvation response factor PHR in plants comprises: transferring the encoding sequence of phosphate starvation response factor PHR into plants; or, modifying the promoter region of PHR downstream target genes, adding a P1BS element, or regulating by an up-regulator that interacts with the phosphate starvation regulator PHR, thereby increasing the expression or activity of the phosphate starvation regulator PHR.

17. The method of claim 16, wherein, the downstream target genes comprise: mycorrhizal symbiosis-specific transcription factor RAM1, mycorrhizal symbiosis-specific transcription factor WRI5A, mycorrhizal symbiosis-specific phosphate transporter PT11 or ammonium transporter AMT3;1.

18. The method of claim 4, wherein, it comprises: knocking out the encoding sequence of phosphate starvation response factor PHR by CRISPR editing system; knocking out the encoding sequence of phosphate starvation response factor PHR by homologous recombination; silencing the encoding sequence of phosphate starvation response factor PHR by a interfering molecule that specifically interfering the expression; or mutating phosphate starvation response factor PHR by loss-of-function mutation; preferably, the downstream target genes comprise: mycorrhizal symbiosis-specific transcription factor RAM1, mycorrhizal symbiosis-specific transcription factor WRI5A, mycorrhizal symbiosis-specific phosphate transporter PT11 or ammonium transporter AMT3;1.

19. The method of claim 8, wherein, the PHR2 comprises: (a) a polypeptide having the amino acid sequence of SEQ ID NO: 2;
(b) a polypeptide derived from (a) with the function of polypeptide (a) by substitution, deletion or addition of one or several amino acid residues in the amino acid sequence of SEQ ID NO: 2;
(c) a polypeptide having more than 50% sequence identity to the amino acid sequence in (a) and the function of polypeptide (a); or
(d) a fragment of SEQ ID NO: 2 with the function of polypeptide (a).

20. The method of claim 14, wherein observing the binding of PHR with the promoter element P1BS of downstream target genes; if the binding is enhanced by the substance, it indicates that the substance can be used for promoting the symbiosis between Gramineous plants and arbuscular mycorrhizal fungi; if the binding is weakened by the substance, it indicates that the substance can be used for inhibiting the symbiosis between Gramineous plants and arbuscular mycorrhizal fungi.


SEQUENCE LISTING (4 sequences)

SEQ ID NO: 1 - DNA, 1281 bp, Oryza sativa
atggagagaataagcaccaatcagctctacaattctggaattccggtgactgtgccatcgcctctgcctgctataccagctaccctggatgaaaacattcccaggattccagatgggcagaatgttccgcgggagagagaattgagaagcacacctatgccacctcatcagaatcagagtactgttgctcctcttcatgggcattttcagtccagtaccgggtctgttgggcctctgcgttcgtcccaggcgataaggttctcttcagtttcaagcaatgagcaatatacaaatgccaatccttacaattctcaaccgccgagtagtgggagttcttcaacgctcaattatggatcacaatatggaggctttgaaccttccttgactgattttccaagagatgctgggccgacgtggtgtcctgatccagttgatggcttgcttggatatacagatgatgtccctgctgggaacaatttgactgaaaacagttctattgcagctggtgatgaacttgccaagcaaagtgaatggtggaatgattttatgaattatgactggaaagatattgataacacagcttgtactgaaactcaaccacaggttggaccagctgcgcaatcatctgtcgcagttcaccaatcagctgcccaacaatcagtttcatctcaatcaggagaaccttctgcagttgctataccctcgccctctggtgcctccaatacctccaactccaagacacgaatgagatggactcctgaacttcatgagcgctttgtagatgctgtcaatctacttggtggcagtgaaaaagctactcccaagggtgtgttaaagctaatgaaggcagacaatttgaccatttatcatgttaaaagtcaccttcagaaatacagaacagctcgatacagaccagaattgtctgaaggttcttcagaaaagaaggcagcctcaaaagaggacataccatcaatagatctgaaaggagggaactttgatctcactgaggcattgcgtctccagttagaactccaaaagaggcttcatgaacagcttgagatccaaagaagtttgcagctgagaattgaggagcaagggaagtgccttcagatgatgctcgagcagcagtgcatacctgggacagacaaggcggtggatgcttcaacctcagcagaaggaacaaagccatcttctgatcttccagaatcttctgccgtgaaggatgttccagagaacagtcagaacggaatagccaaacaaacagaatcaggtgacagataa

SEQ ID NO: 2 - protein, 426 aa, Oryza sativa
MERISTNQLYNSGIPVTVPSPLPAIPATLDENIPRIPDGQNVPRERELRSTPMPPHQNQSTVAPLHGHFQSSTGSVGPLRSSQAIRFSSVSSNEQYTNANPYNSQPPSSGSSSTLNYGSQYGGFEPSLTDFPRDAGPTWCPDPVDGLLGYTDDVPAGNNLTENSSIAAGDELAKQSEWWNDFMNYDWKDIDNTACTETQPQVGPAAQSSVAVHQSAAQQSVSSQSGEPSAVAIPSPSGASNTSNSKTRMRWTPELHERFVDAVNLLGGSEKATPKGVLKLMKADNLTIYHVKSHLQKYRTARYRPELSEGSSEKKAASKEDIPSIDLKGGNFDLTEALRLQLELQKRLHEQLEIQRSLQLRIEEQGKCLQMMLEQQCIPGTDKAVDASTSAEGTKPSSDLPESSAVKDVPENSQNGIAKQTGMRIH

SEQ ID NO: 3 - DNA, 23 bp, Artificial
  target sequence of OsPHR2
cagtccagtaccgggtctgttgg

SEQ ID NO: 4 - DNA, 16 bp, Artificial
  CY5 linker
agccagtggcgataag